(19) United States
(12) Patent Application Publication
(10) Pub. No.: US 2005/0065344 A1
(43) Pub. Date: Mar. 24, 2005

(54) Reagents for N-amination

(76) Inventors: Babu J. Mavunkel, Sunnyvale, CA (US); John J. Perumattam, Los Altos, CA (US); Richland W. Tester, Alameda, CA (US); Sundeep Dugar, San Jose, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/618,573

(22) Filed: Jul. 11, 2003

Related U.S. Application Data
(60) Provisional application No. 60/395,693, filed on Jul. 11, 2002.

(51) Int. Cl. C07D 215/12; C07C 239/20

(52) U.S. Cl. 546/176; 564/300

(57) ABSTRACT

Improved reagents and methods of amination are provided. The reagents are phenyl hydroxylamines containing one nitro and at least one CF₃ substituent on the phenyl moiety.

### CROSS-REFERENCE TO RELATED APPLICATION

This application claims priority from parent application 60/395,693 filed 11 Jul. 2002. The contents of this document is incorporated herein by reference.

### TECHNICAL FIELD

The invention is directed to reagents that are able to aminate nitrogen atoms and to methods to conduct amination using these reagents. More particularly, the invention is directed to a phenyl hydroxylamine which is further substituted with nitro and trifluoromethyl groups.

### BACKGROUND ART

One of the reagents used to couple an amino group to a nitrogen atom recipient is 2,4-dinitro-phenyl-hydroxylamine. This reagent is effective in carrying out the reaction, but has a serious drawback in that it is quite “energetic” and poses an explosion hazard.

Boyles, D. C., et al., Org. Proc. Res. Dev. (2002) 6:230-233 describe a series of alternative reagents where the phenyl hydroxylamine is further substituted by a single nitro group in the para or ortho position and by halo and/or methyl groups elsewhere in the ring. These reagents were used to aminate quinazoline-2,4-diones in order to obtain antibacterial agents for toxicological studies. The contents of this document are incorporated herein by reference.

It has now been found that an improved N-amination reagent can be obtained by preparing substituted (mono-nitrophenyl)hydroxylamines which have, in addition to the nitro substituent, an additional substituent which is trifluoromethyl or CF₃.

### DISCLOSURE OF THE INVENTION

The invention is directed to compounds of the formula 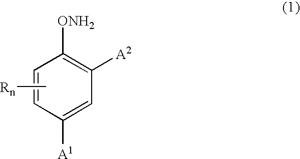
and precursors therefor, wherein at least one of A¹, and A² is nitro, and the other is CF₃, R is halo, alkyl, CN or CF₃ and n is 0-3. These compounds are useful in preparing products, such as those described by Boyle, et al. (supra) that contain an amino group bound to a nitrogen. These compounds may be useful in themselves as antibacterials or modulators of metabolism, or may be intermediates in the synthesis of such compounds.

In another aspect, the invention is directed to methods to aminate nitrogen atoms, especially the N of an indole moiety, which methods comprise contacting a compound, especially an indole, containing a nitrogen which is desired to be aminated with a compound of formula (1) under conditions wherein said amination occurs.

Modes of Carrying Out the Invention

The invention is directed to improved reagents for amination of nitrogen atoms. The reagents are of formula (1) as described above. These reagents can be prepared from either commercially available or synthesized starting materials using standard chemical synthetic methods. Typically, a compound of the formula 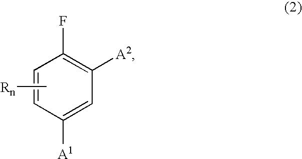
wherein A¹, A², R and n are as defined above, is converted to the phenyl hydroxylamine by displacement of the fluoride substituent. Thus, in one approach, the fluoride is displaced by reaction with an alkyl hydroxyacylimidate, such as ethyl hydroxyacetimidate, in the presence of sodium hydride or another strong base in an appropriate solvent. The resulting intermediate is then treated with a strong hydrolyzing agent such as perchloric acid to yield the corresponding phenyl hydroxylamine.

In the alternative, the compound of formula (2) is reacted with Boc-hydroxylamine to obtain the corresponding —O—NHBoc intermediate which is treated with trifluoroacetic acid to obtain the desired product.

The resulting compounds of formula (1) are then used to treat suitable substrates so as to aminate them. For example, the nitrogen of an indole nucleus may be aminated by treating with the compound of formula (1) in the presence of base and a polar aprotic solvent.

The products of the amination are then useful either as intermediates for further conversion to compounds such as antibacterials, metabolite regulators, and the like. A wide variety of compounds which contain N-N linkages can be prepared using this tool.

In preferred compounds, n is 0, or n is 1 and R represents CF₃ in the position ortho to ONH₂.

However, in addition to CF₃, R may also represent alkyl, halo or CN. “Alkyl” refers to straight chain, branch chain or cyclic substituent containing 1-6 C such as ethyl, n-propyl, cyclohexyl, and the like. Halo refers to fluoro, chloro, bromo or iodo. Chloro is preferred. In general, it is preferred that n=0 or n=1 and, when n=1, R is present in the position ortho to the hydroxylamine substituent.

The following examples are intended to illustrate but not to limit the invention.

### EXAMPLE 1

### Synthesis of 2-Nitro-4-(Trifluoromethyl)Phenylhydroxylamine

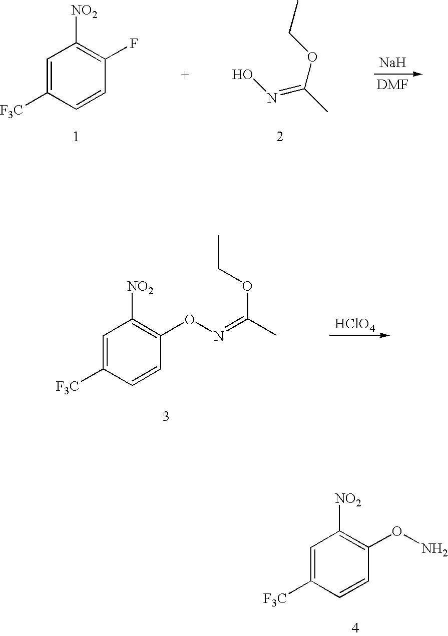

Sodium Hydride 60% dispersion in mineral oil (2.00 g, 49.9 mmol) was added to a stirred solution of ethyl hydroxyacetimidate (2) (4.29 g, 41.6 mmol) in DMF (100 mL) at 0 C under dry nitrogen atmosphere. After stirring at 0 C for 15 minutes, 4-fluoro-2-nitrobenzotrifluoride (1) (8.70 g, 41.6 mmol) was added drop wise. The solution was stirred for an additional hour at 0 C and allowed to slowly warm to room temperature. Ethyl acetate and water were added to quench the reaction. The layers were separated and the organic layer was washed with sat. NaCl solution, dried over sodium sulfate and concentrated. Purification on ISCO chromatography system using ethyl acetate/hexanes gradient gave 10.45 g of 3. NMR (CDCL3) δ s, 1 H, 8.3; d, 1 H, 7.9; d, 1 H, 7.8; q, 2 H, 4.2; s, 3 H, 2.3; t, 3 H, 1.4.

A 70% solution of perchloric acid (20 mL) was added slowly to a stirred solution of 3 (10.45 g, 43.2 mmol) in dioxane (30 mL) at 0 C. The reaction was stirred for an additional 1 hr and ethyl acetate was added. The solution was washed with water, 5% K₂CO₃, dried over sodium sulfate and concentrated. Purification on ISCO chromatography system using ethyl acetate/hexanes gradient gave 6.55 g of 4. NMR (CDCL3) δ s, 1 H, 8.2; d, 1 H, 78.0; d, 1 H, 7.8; 3,2 H, 6.3.

### EXAMPLE 2

### Synthesis of 4-Nitro-2-(Trifluoromethyl)Phenylhydroxylamine

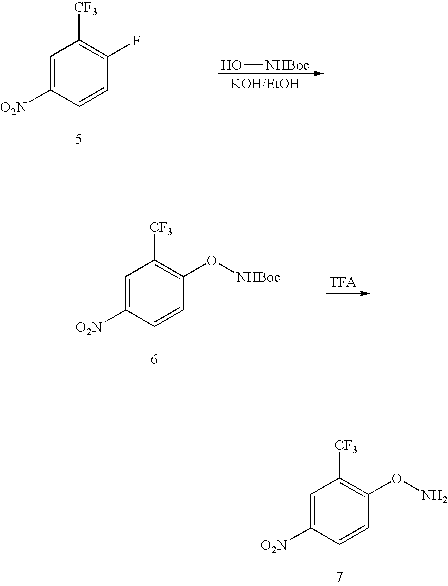

Solid KOH (4.8 g,86.4 mmol) was added to 60 mL of ethanol and stirred until a clear solution resulted. To this solution was added 3.2 g (24.0 mmol) of Boc-hydroxylamine and the reaction mixture cooled to 0° C. To this reaction mixture, a solution of 5.0 g (30.0 mmol) of 2-fluoro-5-nitro-trifluromethylbenzene in 30 ml ethanol was added dropwise (30 min) and stirred at 0° C. for 3 h. Diluted with water and extracted with ethyl acetate, dried and evaporated to give product 6 as a white solid. ¹H NMR (CDCl₃) δ 1.45 (s,9 H), 7.61 (d,1 H), 7.95 (s, 1 H), 8.45 (d, 1 H), 8.61 (s, 1 H).

6 was dissolved in trifluoroacetic acid (30 mL) and the reaction mixture stirred at ambient temperature for 1 h. All starting materials disappeared as monitored by TLC (10% ethylacetate/hexane). trifluoroacetic acid was removed under vacuo. The solids dissolved in ethyl acetate, washed with 10% sodium carbonate, dried and evaporated to give the product as a slightly yellow solid. Recrystallization from 10% hexane in ethyl acetate provided 4.3 g (80%) of phenylhydroxylamine 7 as a white solid. ¹H NMR (CDCl₃) δ 2.25 (s,2 H), 7.42 (d,1 H), 8.41 (d,1 H), 8.51 (s, 1 H).

### EXAMPLE 3

### Amination of Methyl Indole-3-Carboxylate

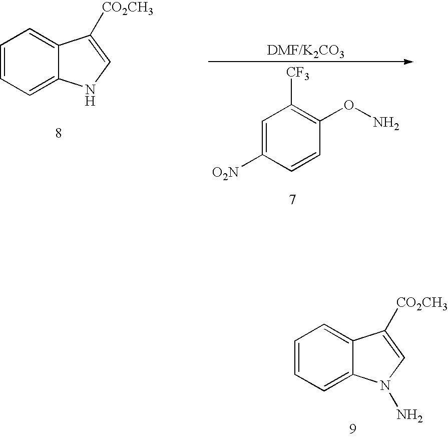

To a solution of indole 8 (175.2 mg, 1.0 mmol) in 3 mL DMF was added finely powdered K₂CO₃ (415.0 mg, 3.0 mmol) and stirred for 1 h. The aminating reagent 7 (288.0 mg, 1.3 mmol) was added all at once and the reaction mixture stirred for 24 h. Diluted with water and the product was extracted with ethyl acetate. The organic layer was dried and evaporated. The product was purified by silica gel column chromatography using 20% ethyl acetate in hexane to obtain 95 mg (50%) of product 9 as white solids MS (M+1 191).

1. A compound of the formula 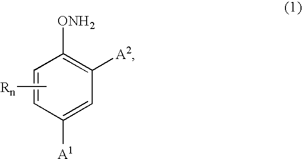
wherein one of A¹ and A² is NO₂ and the other is CF₃, n=0-3 and R is halo, alkyl or CF₃:

2. The compound of claim 1, wherein n=0.

3. The compound of claim 2, which is

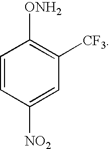

4. The compound of claim 2, which is

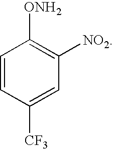

5. The compound of claim 1, wherein n=1.

6. The compound of claim 5, wherein R is ortho to ONH₂.

7. The compound of claim 6, wherein R is CF₃.

8. A method to aminate a nitrogen in a recipient compound which method comprises treating said recipient compound with a compound of formula (1) under conditions wherein said amination can proceed.

9. The method of claim 8, wherein said conditions comprise the presence of base and an appropriate solvent.

10. The method of claim 8, wherein the recipient compound comprises indole.

11. A method to synthesize the compound of claim 1, which method comprises treating a compound of the formula 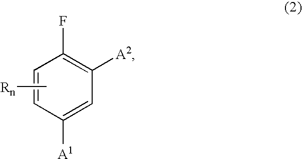
wherein A¹, A², R and n are as defined in claim 1 with an alkyl hydroxylacylamidate or with Boc-hydroxylamine.
